(19) United States
(12) Patent Application Publication
(10) Pub. No.: US 2010/0297313 A1
(43) Pub. Date: Nov. 25, 2010

(54) METHOD AND DEVICE FOR STERILISING A LIQUID

(76) Inventors: Pavel Pavlovitch Koulik, Blaesheim (FR); Aleksandr Zavadtsev, Reutov (RU)

Correspondence Address: BAKER & DANIELS LLP, 300 NORTH MERIDIAN STREET, SUITE 2700, INDIANAPOLIS, IN, 46204, US

(21) Appl. No.: 12/450,256

(22) PCT Filed: Mar. 17, 2008
(86) PCT No.: PCT/IB2008/000619

§ 371 Date: Apr. 21, 2010

(30) Foreign Application Priority Data
Mar. 21, 2007 (EP) 07-005-761.7

(51) Int. Cl. A23L 3/005; A23L 3/01; A23L 3/32; A61L 2/02; C02F 1/48

(52) U.S. Cl. 426/234; 422/22; 210/748.01; 422/307; 422/308

(57) ABSTRACT

A sterilisation process comprising the heating of liquid by waves of electric field having a frequency greater than 1 MHz, at a speed greater than 28° C. per second, to a treatment temperature T between 20° C. and 66° C., and according to the value of the treatment temperature T, exposure of the liquid to an alternating electric field in pulses immediately or slightly after heating of the liquid.

The invention relates to a process for sterilisation or pasteurisation of a liquid, especially a water-based liquid or a liquid containing water, and/or bodies or solid objects in contact with the liquid, and a device for carrying out the process.

“Sterilisation” is understood as the destruction or neutralisation of microorganisms, such as yeasts, moulds, bacteria and viruses, selectively or across a broad-spectrum, i.e. targeting just one or several types of microorganisms, or essentially all types of microorganisms contained in the liquid or on the surfaces of bodies or solid objects in contact with the liquid. In the present application, the notion of sterilisation also covers what is conventionally known as pasteurisation.

In particular, the term sterilisation is used in the present invention to qualify a process of selective or non-selective destruction or neutralisation of microorganisms preferably to below a threshold of 100 microorganisms/ml remaining in the liquid to be sterilised. The invention is mainly, though not exclusively, applicable to food, pharmaceutical and medical, biophysical and biochemical fields, and to water supply systems.

By way of example liquids to be sterilised can comprise contaminated water, wastewater, sewage water, stagnant water, blood and components of blood, pharmaceutical preparations, drinks or food products such as beer, mineral water, flavoured water, milk and dairy products, tea and others.

A conventionally used sterilisation process is by heat treatment (pasteurisation), over a certain time, at a sufficient temperature to destroy microorganisms. Conventional sterilisation (pasteurisation) temperatures are between 90 and 120° C. These processes have the disadvantage that they alter the properties of the sterilised liquids, for example by destroying vitamins. Also, the high temperatures prevent the use of such processes for the sterilisation of liquids in containers made of plastic, such as PET bottles.

Patent WO 02/34075 A1 discloses a process for the sterilisation of a liquid and/or a solid object in contact with this liquid by heating simultaneously with action of an electric field and acoustic vibrations. According to this document, this process would allow the sterilisation of a liquid, and of the prior closed container which contains it, at a critical temperature T_{c} less than the thermal sterilisation (pasteurisation) temperature Tₜ.

However, in practice, this process does not substantially lower the critical temperature T, due to the fact that the heating of the liquid is actually not effective. Heating is carried out by the application of an electric field, with an amplitude at a level of 1000 V/cm and the frequency of the electric field being in the frequency ranges of 10⁷ Hz or 10⁹ Hz. However, the structure of microorganisms is not sensitive to such an electric field of overly low amplitude and overly high frequency. On applying the conditions described in WO 02/34075 A1, it seems that it is not possible to lower the sterilisation (pasteurisation) temperature to below 70° C.

Other documents of the prior art, specifically patents U.S. Pat. Nos. 4,695,472, 5,048,404 and the article “A Continuous Treatment System for Inactivating Microorganisms with Pulsed Electric Fields” mention pasteurisation of food products at relatively low temperatures. In U.S. Pat. No. 4,695,472 the sterilisation of liquid foodstuffs at treatment temperatures of at least 45° C. is described. The liquid is heated and subjected to one or more electric field pulses with an amplitude between 5.000 and 12.000 V/cm for currents of at least 12 A/cm² and duration between 5 and 100 microseconds. In these conditions, it is question of a process delaying growth of the microorganisms typically by ten days, and not of a process for destruction of the microorganisms, that is, sterilisation of the product. Also, the creation of electric field pulses is accompanied by an electric current causing additional heating of the product, the power density in the cited examples reaching values of up to 6 W/cm³. A disadvantage of this process is that the efficacy of the heating is diminished due to the creation of preferrential current passages (“pinch” effect), accompanied by the risk of excessive local heating and even breakdown, possibly resulting in alteration of the physico-chemical properties of the liquid to be treated.

The process described in U.S. Pat. No. 4,695,472 does not allow the sterilisation of liquids enclosed in containers of size usual in the food industry, not only due to the problems mentioned above, but also due to the fact that the proposed amplitude of the electric field, applied to a bottle of some ten centimetres in diameter, would need very high voltages, difficult to generate and to apply homogeneously.

The process' of irreversible electroporation can be considered as a process enabling in principle low-temperature sterilisation of aqueous liquids (for example at 20° C.) by subjecting liquid to repeated electric field pulses of 10-20 KV/cm. In the case of sterilisation of drinks containers of 0.5-1.5 litre, this would mean that voltages exceeding 10-20 10⁶V would have to be provided, which is not feasible under industrial conditions.

In light of the above, an aim of the invention is to provide a process for sterilisation or pasteurisation of liquids, which is effective and reliable, which does not alter or only slightly alters the properties of the liquid. An aim is also to provide a device for carrying out such a process.

It is advantageous to provide a process for sterilisation of liquid which does not heat the liquid, even locally, above 70° C., preferably not above 65° C.

It is advantageous to provide an sterilisation process which is economical and simple to control and carry out.

It is advantageous to provide an efficient process enabling effective and reliable sterilisation of liquid hermetically enclosed in containers, especially containers of current sizes in the food industry, including containers made of plastic or other materials not supporting high temperatures.

Aims of the invention are realised by a sterilisation process according to claim 1, and a device for carrying out a sterilisation process, according to claim 11.

The sterilisation process according to the present invention comprises heating the liquid by electric field waves having a frequency greater than 1 MHz, at a speed greater than 28° C. per second, to a treatment temperature T between 20° C. and 66° C., and according to the value of the treatment temperature T, exposure of the liquid to an alternating electric field in pulses immediately or slightly after heating of the liquid, the amplitude E of the electric field in V/cm being selected such that the equation:

C(T)≦log(E+1)≦B(T)

is satisfied for the values:

B(T)=−2.340×10⁻⁵T³+1.290×10⁻³T²−3.110×10⁻²T+5.0

C(T)=−4.503×10⁻⁵T³+2.888×10⁻³T₂−5.900×10⁻²T+4.0

where T is the treatment temperature in Celsius.

Surprisingly, the inventors found that by reheating liquid very rapidly, at a speed greater than 28° C. per second, the electric field to be applied to destroy the microorganisms can be considerably reduced. Thus, at treatment temperature values of 64 to 66° C., the amplitude of the electric field can even be zero. In other words, effective and reliable pasteurisation of the liquid does not require any exposure to an electric field for a treatment temperature over 64° C., and for lower temperatures, exposure to a field of amplitude much less than what is conventionally proposed.

Due to the importance of the speed of heating on the efficacy of pasteurisation, uniform heating in volume is important to ensure that the entire volume of the liquid is subjected to rapid heating. For this purpose, the liquid is preferably agitated or turbulised and reheating in volume is carried out by high-frequency waves or microwaves. Heating by HF waves or microwaves makes it possible to obtain heating by agitation of the water molecules, on minimising ohmic heating by electric current, to prevent “pinch” effect problems causing non-uniform heating. The frequencies of this radiation are preferably more than 1000 kHz.

To process hermetically sealed containers it is advantageous to use alternating electric fields at a frequency greater than 100 kHz, but less than 1000 kHz. As the lipid membrane of the microorganism has a certain inertia, it does not react to electroporation above around 1000 kHz. In practice, application of an electric field of frequency less than 100 kHz will be accompanied by an electric current which heats the aqueous solution, creating at the limit local breakdown zones, which is undesirable.

To prevent overheating and breakdown zones, the electric field is applied in pulses. The amplitude of the electric field and the duration of a pulse of electric field are preferably adjusted to avoid the appearance of breakdown in the liquid to be sterilised. In the case of a plurality of electric field pulses, the total duration of the electric field pulses and their frequency are preferably selected so as to avoid heating the liquid to be treated by more than a few degrees.

According to the invention, the total calorific energy provided to the aqueous solution by the electric field pulses is preferably less than 0.05 J/cm³ and the repetition frequency of the alternating electric field pulses on each portion of the liquid to be treated is preferably between 10 and 100 Hz.

It is useful to make a pause between the heating step and the step of application of the electric field pulse(s). This pause is useful to better make uniform the temperature field in the liquid to be sterilised so that all the zones of the liquid, including those of the layers bordering on the liquid-solid interfaces of the container, acquire essentially the same temperature before application of the electric field.

The parameters of the thermal pulse and of the electric field according to the present invention depend on the thermodynamic of the evolution of the molecular states of the membrane surrounding the microorganism and responsible for its vitality, when this membrane is immersed in liquid containing water.

The qualitative understanding of the role of the temperature and of the electric field in the evolution of the molecular states in the membrane surrounding the microorganisms and responsible for its vitality is based on studying the behaviour of the structures of the lipid molecules in contact with the clusters of water when the membrane is immersed in an aqueous solution and subjected to an electric field. In general, the membrane is subjected to the formation of pores (“poration”). These pores form and close up sporadically. When the electric field is zero, the increase in temperature causes irregularities in the structure of the lipid molecules of the cellular membrane of the microorganisms due to the change in form of the “tails” of lipid molecules. If a pore forms these changes in form cause phase transformations which stimulate an increase in the size (and possibly also the number) of pores until stability is lost, or the membrane tears. Normally, these transitions can take place from and above a temperature close to 70° C. This phase transition causes an increase in the diameter of the pore, tearing of the membrane and the “death” of the microorganism. Yet if the temperature rises slowly the phase transition is delayed, the membrane resists this increase in temperature, adapts its molecular morphology to a metastable state, and only at higher temperatures (around 100° C.), does the phase transition take place, accompanied by the tearing of the membrane and therefore the “death” of the microorganism. The values of 70° C. and 100° C. are only average values. These values depend on the nature of the microorganism. As a function of the nature of the microorganism concerned, these values can vary between 65 and 75° C. and between 95 and 135° C. respectively.

The slow increase in temperature corresponds to classic thermal destruction (sterilisation). For rates of temperature increase in the order of 1° C. per second and less, according to current practice, this produces classic metastable sterilisation.

On the contrary, for rates of temperature increase of over 28° C. per second, preferably greater than 30° C. per second, any adaptation of the molecular morphology of the microorganisms to a metastable state is avoided.

Thermal stresses on the membranes of the microorganisms due to the very rapid increase in temperature of the liquid add stresses due to the effects of the alternating electric field, the frequency of which is selected to oscillate the effects of stress on the membranes and consequently amplify the maximum local stresses which these membranes undergo. This combination allows a better concentration of the energy of the electric field on destruction of the microorganisms by electroporation, minimising the electric energy loss in heat and therefore the electric power necessary for irreversible destruction of the microorganisms. This allows the treatment of larger volumes and allows to more easily avoid problems of breakdown and local heating which can alter the properties of the liquid to be sterilised.

An important advantage of the present invention is therefore to be able to perform, at temperatures under 66° C., and with an electric field of low amplitude relative to conventional processes, irreversible collective electroporation operations on cells found in large numbers in an aqueous solution, in particular inside a hermetically sealed container.

This allows the pasteurisation of liquids at a temperature at which containers made of plastic materials do not deform and the physico-chemical properties of the liquid are not modified/degraded.

The sterilisation process according to the invention can advantageously be carried out selectively, since for each sort of microorganism the specific parameters (amplitude, oscillation frequency, pulse frequency, pulse duration) for the destruction of said microorganism can be selected. This makes it possible to better target the destruction of harmful microorganisms, and if necessary, to not destroy a certain quantity of useful microorganisms.

The sterilisation process according to the invention can advantageously be applied to continuous flow, pulsed flow, containers filled with liquid to be sterilised, or even containers filled with liquid and in an aqueous solution, enabling also the sterilisation of the internal and external surfaces of the containers.

The present invention can be applied to any solid body made of dielectric material, in particular a polymeric material. Solid bodies can be in the form of hermetically sealed containers containing an aqueous solution, in particular in the form of containers made of plastic, such as PET bottles or supple plastic sachets, or even glass bottles.

The practical conclusion of this analysis is that a first measure for decreasing the treatment temperature is to conduct rapid heating of the liquid containing the microorganisms, preferably at a rate of over 30° C. per second and more advantageously from 30 to 40° C. per second. This makes it possible to obtain tearing of the membrane of the microorganisms at temperatures lower than conventional pasteurisation temperatures and with electric fields much weaker than the fields proposed in the prior art, even zero for a treatment temperature above 64° C.

The interaction of a low-frequency electric field, in particular between 100 kHz and 1000 kHz, with the dipoles of the “tails” of the lipid molecules concentrated at the surface of the pore, causes displacement of the threshold of the phase transition temperature towards low temperatures. The greater the amplitude of the electric field, the more the threshold moves down. This means that the lethal temperature threshold for microorganisms towards can be decreased towards and down to ambient temperature. The amplitude of electric field necessary for killing a microorganism (by electroporation) at ambient temperature (20° C.) is of the order of 10⁴ at 2×10⁴ V/cm. It is important to emphasise that this concerns the amplitude of the local electric field, that is to say, in the liquid to be treated or at the liquid-membrane interface.

The device for executing the sterilisation process comprises a heating station with a liquid-heating system, an electric field generation station of with a system for the generation of electric fields by pulses, and device for transport of the liquid to be treated comprising a conduit able to transport liquid passing through the heating and electric field application stations, the heating system being configured to heat liquid passing through the heating station at a rate greater than 28° C. per second. The system for generation of electric field by pulses is configured to generate an alternating electric field with an oscillation frequency between 100 kHz and 1000 kHz.

The device preferably comprises a cooling station downstream of the station for generation of electric field, through which the transport device passes, in order to rapididly cool the liquid to be treated.

According to one variant, the system for generation of electric field pulses comprises electrodes arranged on either side of a section of passage of the conduit and capable of generating an electric field transversal to this section.

According to another variant, the system for generation of electric field pulses comprises an inductor with one or more primary windings arranged toroidally about a section of passage of the conduit and capable of generating an electric field essentially longitudinal to this section.

The device can also comprise an electric field sensor in the application zone of the electric field and temperature sensors along the transport device, upstream of, downstream of and in the heating station.

The transport device can comprise a pump system and transport liquid for transporting containers containing the liquid to be treated along the conduit, and a return circuit for returning the transport liquid from an outlet to an inlet of the transport device.

The conduit of the device can have parts with different cross-sections of passage, intended to vary the flow speed of the liquid.

The device can advantageously be used for the decontamination of blood or a liquid component of blood contained in hermetically sealed supple containers or for sterilisation of drinks or liquid food products contained in hermetically sealed containers such as bottles made of glass or plastic.

Other aims and advantageous characteristics of the invention will emerge from the following detailed description, by way of illustration, with reference to the attached diagrams, in which:

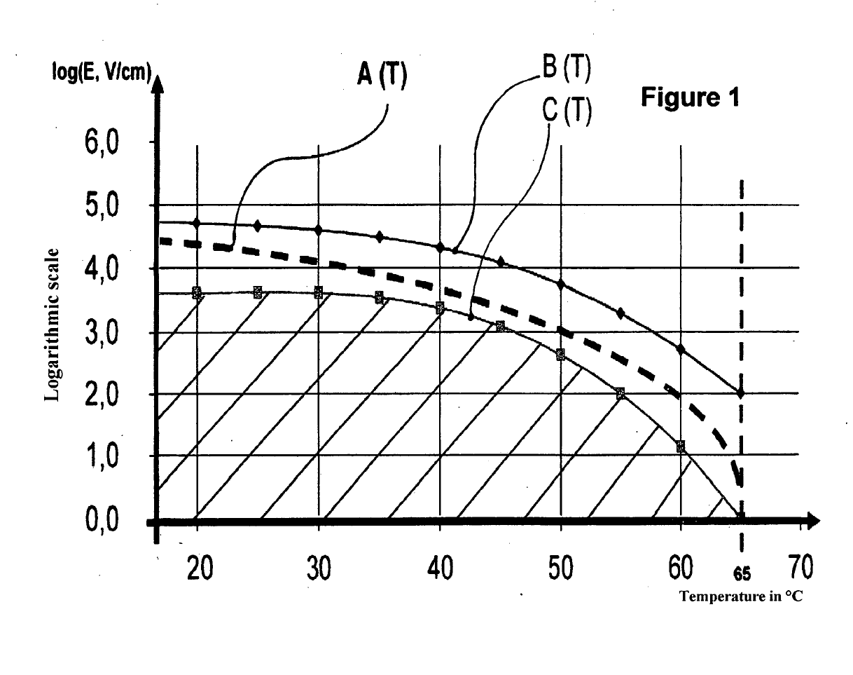

FIG. 1 shows a graph illustrating the relation between the treatment temperature and the amplitude of the electric field according to the invention;

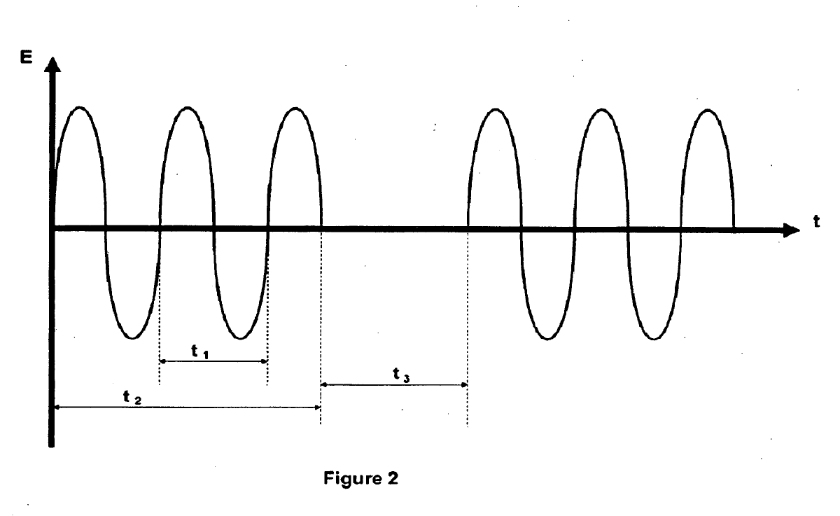

FIG. 2 shows a graph illustrating electric field pulses according to the invention;

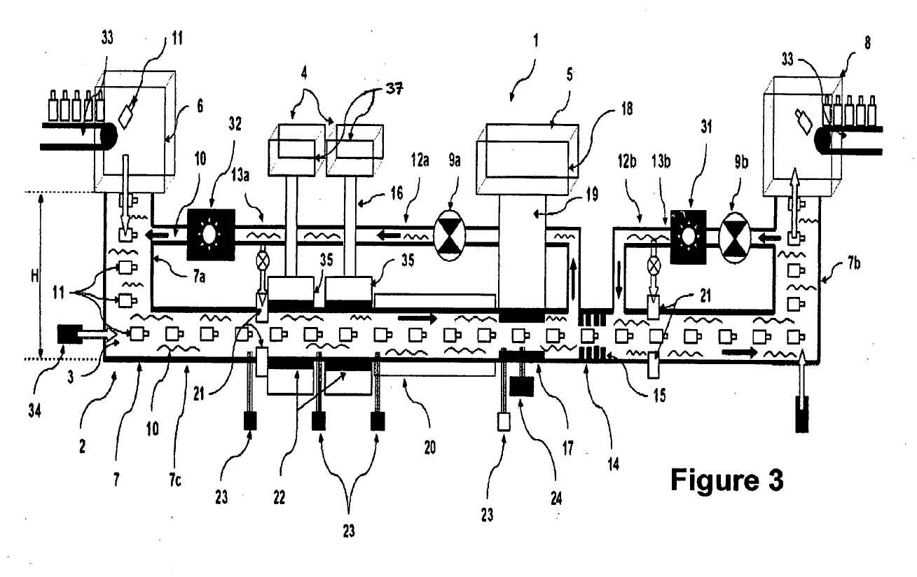

FIG. 3 shows a device for carrying out a sterilisation process according to an embodiment of the present invention;

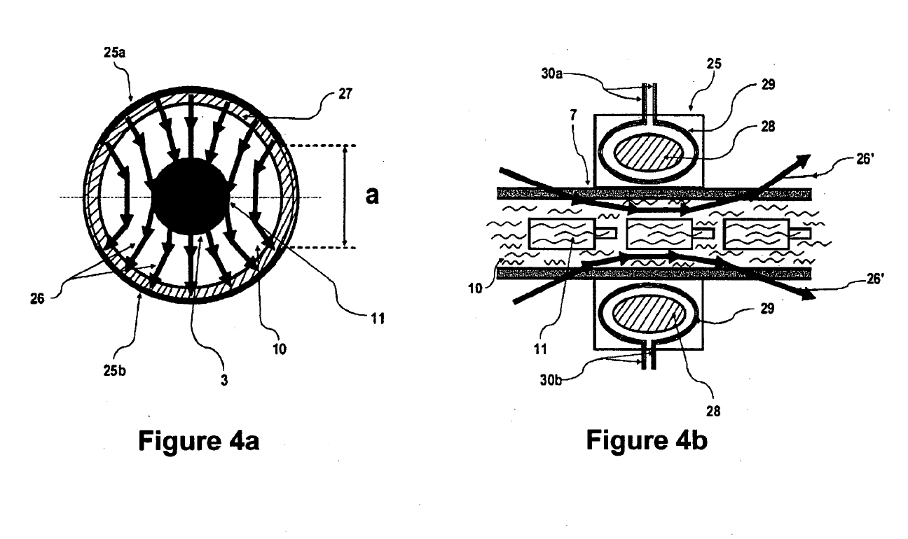

FIG. 4a shows a electric field distributor device according to a first embodiment; and

FIG. 4b shows a electric field distributor device according to a second embodiment.

The sterilisation process according to the present invention comprises heating the liquid to be treated by an electric field having a frequency greater than 1 MHz, at a speed greater than 28° C. per second, to a treatment temperature T between 20° C. and 66° C. According to the value of the treatment temperature T, the liquid is exposed to an alternating electric field in pulses immediately or slightly after heating of the liquid, the amplitude E of the electric field in V/cm being selected such that the empirical equation:

C(T)≦log(E+1)≦B(T)

is satisfied for the values:

B(T)=−2.340×10⁻⁵T³+1.290×10⁻³T²−3.110×10⁻²T+5.0

C(T)=−4.503×10⁻⁵T³+2.888×10⁻³T²−5.900×10⁻²T+4.0

where T is the treatment temperature in Celsius.

This relation is illustrated by the graph of FIG. 1.

B(T) represents the upper limit of the amplitude of the electric field reasonably necessary under industrial pasteurisation conditions of water-based products according to the present invention.

C(T) represents the lower limit of the amplitude of the electric field below which there is not destruction of all the typical microorganisms representing a danger for the quality and the conservation of the product or for the health of the consumer or of the individual (hatched zone in FIG. 1).

A(T) represents the lower limit of the amplitude of electric field below which, according to the present invention, pasteurisation of a water-based product containing typical microorganisms representing a danger to the quality and conservation of the product or for the health of the consumer or of the individual does not take place.

For example, the value of the electric field necessary for pasteurising la iquid according to A(T) is:

E≈0 V/cm, when T=65° C.

E≈10² V/cm, when T=60° C.

E≈10³ V/cm, when T=50° C.

E≈5.10³ V/cm, when T=40° C.

E≈10⁴ V/cm, when T=30° C.

E≈5.10⁴ V/cm, when T=20° C.

It is evident that this relation gives only an initial estimation, which can be specified empirically as a function of the microorganisms (cells) to be destroyed and the properties of the liquid.

The aspect of pulses of the alternating electric field is illustrated in FIG. 2 where the times t₁, t₂ and t₃ are indicated.

Oscillation of the electric field is preferably essentially sinusoidal, but can take another form.

The characteristics and form of the alternating electric field pulses are configured to maximise electroporation of the membranes of the microorganisms and reduce the generation of electric current lost to heat. For this purpose, the period t₁ of an oscillation of the electric field preferably has a value

t₁>1 μs (10⁻⁶ seconds)

Below this duration, the microorganisms are insensitive to the oscillations of the electric field.

For a constant amplitude of electric field, the greater t₁ is, the more intense are the losses of current due to ohmic heating accompanying passage of the oscillating electric current through the heated medium, given the finite electrical resistivity of the medium. In the case of heating containers made of plastic filled with drink by high-frequency currents, in order to minimise these losses, it is very advantageous to limit the frequency to 100 kHz, or t₁ to 10 μs, preferably at 5 μs.

There is for t₁ is therefore the limiting condition:

1 μs<t₁<10 μs.

The duration t₂ of a pulse of oscillating electric field is greater than the period t₁ of an oscillation of the electric field:

t₂>t₁.

The upper value of t₂ is determined by total heating of the thermal perturbation zones due to the fact that the electrical resistance of the electrolytes —drinks are a particular example —decreases with the increase in temperature. The electric current in this case will always be concentrated in more or less cylindrical zones oriented along the vector of the electric field. These zones consequently contract rapidly, stimulated by “pinch” effects. The temperature in these zones rises exponentially, resulting in unacceptable local heating, or even breakdowns. These constraints result in the limiting relation for t₂:

t₂<c ·dT ·R/E²

where c, dT, R, E are respectively specific heat, limit temperature gap, resistivity of the medium, and amplitude of the electric field.

Taking into account the experimental fact that the electrical resistance of an aqueous medium such as a drink does not exceed 10 Ohm.m and that c=4 megajoules/m³ degree, for dT<0.5 degrees Celcius and E=1000 kV/m, there is:

t₂<20 μs.

The duration t₃ is the time lapse between two pulses of electric field. It is preferably more than the time of compensation of the ohmic heating perturbations by the pulses of hydrodynamic turbulence.

If v is the characteristic speed of hydrodynamic instabilities and L is their amplitude, the compensation condition is:

t₃>L/v

In the case of pasteurisation of sealed bottles filled with drink, according to the present invention, there is L>0.003 m and v<1 m/s, giving t₃>0.001 s.

The upper limit for t₃ is given by the condition of having at least one pulse per treated container. In this case t₃<LL/vv, where LL is the characteristic dimension of the container in the direction of its movement across the electric field, and vv its speed.

For a typical case of pasteurisation of bottles of 0.5 l, LL=0.3 m and vv>1 m/s, there is:

t₃<0.3 s.

If a flow of liquid is treated, t₃<LLL/vvv where LLL is the length of the zone of application of the electric field and vvv is the speed of flow through this zone.

For a typical case where LLL=0.3 m and vvv>1 m/s, there is:

t₃<0.3 s.

In the sterilisation process according to the invention, heating of the liquid can take place simultaneously with the pulse or pulses of electric field. In practice, it is more advantageous to first subject the liquid to the heating pulse, and to then apply the pulse or pulses of electric field. This pause is useful for better evening out the temperature field in the liquid to be sterilised such that all the zones of the liquid, including those of the layers bordering the liquid-solid interfaces of the container, acquire essentially the same temperature prior to application of the electric field.

If x is the characteristic thickness of the boundary layer (at most 0.3 mm), the duration of the pause tₚ is preferably greater than:

tₚ=(d·c·x²)/z

where d, c and z are respectively the density, thermal capacity and thermal conductivity of the liquid to be sterilised. For the majority of applications the duration of this pause does not exceed 1 or 2 seconds.

For some applications it is advantageous to space the zone of action of the thermal pulse from that of the electric field pulse. For example, a transit zone can be inserted between the two, where the electric field is zero or negligible and where the temperature field evens out in the volume of the liquid such that the difference in temperature between the central and peripheral parts of the liquid does not exceed one degree. The liquid to be treated passes through this transit zone during the above-mentioned pause between the heating of the liquid and the application of the electric field.

FIG. 3 illustrates a scheme of the device for implementing the process according to the present invention.

The device 1 comprises a transport system 2 of the liquid to be treated 3, a station for the heating in volume 4 of the liquid to be treated and a station of application of an electric field in pulses 5.

The transport system 2 comprises an inlet station 6, a transport conduit 7, and an outlet station 8. The containers can be guided by a standard conveyor 33 and deposited onto a bucket chain (or any other equivalent mechanism) in a column part 7a of the conduit 7.

The transport system can also comprise a pumping system 9a, 9b, for circulation of the liquid to be treated in the case of treatment of a continuous flux of liquids, or for circulation of a transport liquid 10 in which hermetic containers 11 containing the liquid to be treated 3 are immersed. The transport system can advantageously include a hot circuit 12a and a cold circuit 12b, each fitted with a pumping system 9a, 9b and system of recirculation of the transport liquid. The hot circuit 12a transports the containers across the stations for heating and application of the electric field and returns the transport liquid via a return conduit 13a to the transport conduit 7 in the proximity of the inlet station. The cold circuit 12b also has a pumping system 9b and a return conduit 13b interconnecting with the transport conduit 7 between a position in the proximity of the outlet station 8 and an interface 14 separating the hot and cold circuits.

The interface 14 advantageously comprises seals 15 in the form of a plurality of flexible juxtaposed walls, for example made of rubber, comprising openings adapting to the profile of the container to be treated. In this way the container participates in creation of sealing between the hot and cold circuits.

The hot and cold circuits can also comprise heat exchangers 31 and 32 on the return conduits, for recovering heat from the transport liquid and/or the liquid to be treated.

The cold circuit rapidly lowers down the temperature of the liquid to be treated to preserve the properties of the liquid and, if necessary, reduce the problems of deformation of containers made of plastic materials.

The heating station 4 comprises a system for generating thermal pulses 35 fed by a thermal energy generator 37. The thermal generator can be, for example, in the form of a generator of high-frequency electric field operating at a frequency greater than 1 MHz or a microwave generator. The energy is transferred from the generator 37 to the system 35 by means of a coaxial cable or a waveguide 16. It is possible to provide several generators arranged in a juxtaposed manner along the transport conduit 7.

The station of application of an electric field 5 comprises a bipolar oscillating electric field pulse distributor 17 connected to a bipolar oscillating electric field pulses generator 18 by means of a coaxial cable 19.

The stations of thermal pulses 4 and of application of the electric field 5 are separated by a thermally insulated transit section of the conduit 20, creating a pause between thermal treatment and electric pulse treatment. This pause advantageously enables uniform distribution of the temperature field in the liquid to be treated and on the surfaces of the solid bodies on contact therewith.

In the embodiment of FIG. 3, the liquid to be sterilised is contained in containers 11 immersed in a transport liquid 10 flowing in the conduit 7 for transporting containers. The containers can be, for example, bottles made of plastic filled, for example, with drink or a liquid foodstuff.

Once they are lifted in the outlet column part of the conduit 7b, the containers can be evacuated by a ram or other mechanism onto a conveyor 33.

It is also possible to transport the containers containing the liquid to be sterilised via a heating station and a station of application of the electric field by means other than liquid in a conduit, for example by a pressurised gas flow in a conduit (the pressure of the gas being selected so as to compensate the pressure inside the container, thus avoiding any deformation of the container due to heating) or by a mechanical transport mechanism such as a conveyor system. However, a transport system by fluid has the advantage of enabling a good uniformity in temperature distribution around the container during heating and during the pause prior to application of the electric field. The use of a transport liquid having dielectric properties similar to those of the liquid to be sterilised advantageously allows good control of the heating of the liquid to be sterilised as well as of the application of the local electric field in the liquid to be sterilised.

The containers, made of dielectric material, can be in the form of rigid containers, such as glass bottles or made of plastic (for example PET), or in the form of supple containers, such as sachets made of plastic (polypropylene, PET, or other polymers).

The liquid to be sterilised can also flow directly in the conduit of the device passing through the heating and application stations of the electric field.

Agitation devices 21 can be added to the system to agitate the liquids and, if necessary, the bodies in a transport liquid. In one variant, the agitation device creates turbulence in the liquid flowing in the conduit, thereby making the temperature field in the liquid uniform. Containers transported in the conduit can also be agitated or rotated, for example by controlling currents in the transport liquid, so as to make uniform the liquid to be treated inside the containers.

Tubes made of dielectric material (quartz, for example) 22 are installed in the conduit to ensure passage of the electric field serving for the heating of the liquid inside the conduit.

Temperature sensors 23 are arranged all along the conduit for measuring the temperature of the liquid at the entry to the station for generation of thermal pulses, in the heating zone, at the exit of this zone and at the exit of the transit section of the conduit.

An electric field sensor 24 is arranged in the zone of application of the electric field.

In an embodiment of the device, a mechanism is provided to ensure a variable displacement speed of the solid bodies during their passage in the conduit, for example, by changing the cross-section (diameter) of the conduit to vary the speed of the flux of the transport liquid.

An electric field distributor device, according to a first variant, is shown in FIG. 4a. In this variant, the distributor comprises electrodes 25a, 25b located on either side of the conduit to assure the passage of alternating electric field pulses of frequency between 100 kHz and 1000 kHz transversally through the conduit 7 (FIG. 3), as illustrated by the field lines 26.

In particular, the electric field passes from the upper electrode 25a to the lower electrode 25b, the two electrodes being installed inside a tube 27 (quartz, for example), hermetically integrated in the conduit in which the liquid 3 and 10 flows. The distance “a” between the electrodes can be optimised empirically to ensure the best possible uniformity of the electric transversal field in the volume of the containers 11. If the distance a is for example of the order of 4 cm, to produce an amplitude of effective electric field of 1-3 kV/cm, there must be a potential difference between the electrodes of the order of 400-1200 kV.

FIG. 4b shows an electric field distributor device according to a second variant. In this variant, the electric field pulses are created by an induction system and the lines of electric field 26′ are essentially longitudinal. The conduit 7, filled with water such as transport liquid 10 transporting containers 11, such as bottles containing liquid to be sterilised, passes through a body of the induction system 25. The electric field distributor device is equipped with a core 28 and one or more primary windings 29 attached to a feed via connections 30a, 30b. The quantity of primary windings can be determined empirically, for example by measuring the electric field present in the transport liquid.

In the embodiment of FIG. 3 the containers 11 are immersed to a depth H in a column part 7a of the transport conduit 7 filled with transport liquid 10.

The column of transport liquid exerts an external pressure which tends to compensate the internal pressure during heating of the liquid to be treated according to formula (2) which determines the height H of the column corresponding to the temperature T>T₁.

H×d×g=(T₂/T₁)×P₁−C+V_{P}+V_{S} (2)

where:
“H” is the height of the column of liquid in which the containers to be treated are immersed;
“d” is the density of the external liquid;
“g” is the local acceleration of gravity;
“P₀” is the initial pressure of the compressible liquid in the container on entry to the device;
“Vs” is the difference between the saturated vapour pressure of the incompressible liquid at temperatures T₂ and T₁. For water, at T₁=20° C. for example, the saturated vapour pressure is minimal and Vₛ is practically equal to the saturated vapour pressure of water at the temperature T₂. For example, if T₂=65° C., then Vs=0.25 bar;
“C” is equal to (k×Vᵥ) where k is the coefficient of volumic elasticity of the material of the container at the temperature T₂ and Vᵥ is the volumic deformation;
“Vₚ” is the variation in internal pressure due to variation in saturation of the incompressible liquid by the compressible liquid. Vₚ is measured in a non-deformable container (for example glass) of the same shape and volume as the treated container, as the difference in pressure between the real manometric pressure at the temperature t₂ and the pressure P₂=P₀×(T₂/T₁). For drinks not saturated in CO₂, such as for example flavoured water or milk, Vₚ is close to zero. The compensation is total when C=0.

The depth H can be decreased by increasing the density d of the external liquid medium in which the containers are immersed. In particular, solid bodies of small dimension p (p must be much less than the characteristic dimension of the container) but of density greater than that of the liquid, for example in the form of powder, can be added to this liquid. This measure will be effective only when the pressure exerted by the solid bodies is equal in all directions. For this, the solid bodies must be provided with chaotic movement of which the average speed is greater than the square root of gp where:

“g” is the local acceleration of gravity
“p” is the dimension of the solid bodies
and their specific quantity n (quantity of solid body per unit of volume) must correspond to the desired increase in density d.

To satisfy this condition, the force of gravity of the solid body of mass m, i.e. mg, must be less than the force F exerted by this body on any wall due to its inertia. If v is the speed of chaotic movement, the following order of magnitude can be obtained for F: F=m×(v/t), where t=d/v, then F=(mv²)/d. Therefore F>>mg, therefore v>>(gd)^{(1/2)}.

If bottles are treated sequentially and in the direction of their length, one behind the other, a ram 34 sends the bottles into the horizontal part of conduit 7c.

The present invention may be used in the medical and pharmaceutical fields, especially for selective decontamination of microorganisms in blood or in components of blood or in other pharmaceutical preparations. It can also be used for the destruction of colonies of legionelloses in waste water.

The process and the device proposed in the present invention can advantageously be used in the food industry for decontamination (pasteurisation, sterilisation) of water-based food products or those containing water, such as fruit juices, beers, flavoured water, natural mineral water, milk, dairy products and other drinks and liquid foodstuffs.

The present invention is of interest for applications in the field of hygiene, in particular for disinfecting waste water, sewage water, and stagnant water.

### EXAMPLES

1. Decontamination of 0.51 PET bottles, filled with freshly squeezed orange juice contaminated by “Byssochlamys nivea” microorganisms. Treatment was carried out on a device of the type illustrated in FIG. 3:
Initial concentration of microorganisms: from 3.6 to 4.2 10⁵ units/ml;
Quantity of bottles treated for each cycle: 10;
Initial temperature: 20° C.;
Duration of treatment: 3 s (passage through horizontal conduit);
Heating: microwave 1 GHz, power 180 kW (35° C./s) and 45 kW (9° C./s);
Application of the electric field:
   Frequency of oscillation of the electric field: 180 kHz;
   Duration of a batch of oscillations: ca. 0.02 ms;
   Frequency of batches of oscillations: 15 Hz;
   t₁=6 μs, t₂=20 μs, t₃=0.05 s;
   Quantity of pulses: 12 for 180 kW and respectively 35 and 48 pulses for 45 kW;
Productivity, linear speed of bottles: 0.4 m/s for 180 kW and 0.1 m/s for 45 kW. Length of the application zone of the field: 0.3 m; duration of application of electric field pulses: 0.75 s;

Results:

| | Speed of | Treatment | Residual | Residual |
|---|---|---|---|---|
| Electric | temperature | temperature | concentration | concentration 2 |
| field | increase in | in | after tests | months after tests |
| (V/cm) | ° C./s | ° C., +/−1° C. | (units/ml) | (units/ml) |
| 0 | 9 | 80 | <1 | <1 in 80% of cases |
| 0 | 9 | 65 | From 5 | — |
| | | | to 20 | |
| 0 | 35 | 65 | <1 | <1 in 100% of cases |
| 0 | 35 | 62 | From 120 | — |
| | | | to 1500 | |
| 30 | 35 | 62 | <1 | <1 in 95% of cases |
| 0 | 35 | 60 | ca. 10⁴ | — |
| 100 | 35 | 60 | <1 | <1 in 100% of cases |
| 0 | 35 | 55 | ca. 3-4 · 10⁵ | — |
| 600 | 35 | 55 | <1 | <1 in 100% of cases |

2. Selective decontamination of 0.51 PET bottles, filled with apple juice and contaminated by Saccharomyces cerevisiae yeasts and Aspergillus Niger mould. Treatment was carried out on a device of the type illustrated in FIG. 3:
Initial concentration of Saccharomyces cerevisiae: 1.2-3.1. 10⁵ units/ml;
Initial concentration of Aspergillus niger 1.5-4.2. 10⁵ units/ml;
Quantity of bottles treated for each cycle: 10;
Initial temperature: 20° C.;
Duration of treatment: 3 s (passage through horizontal conduit);
Heating: microwave 1 GHz, power 180 kW (35° C./s) and 45 kW (9° C./s);
Application of the electric field:
   Frequency of oscillation of the electric field: 180 kHz;
   Duration of a batch of oscillations: ca. 0.02 ms;
   Frequency of batches of oscillations: 15 Hz;
   t₁=6 μs, t₂=20 μs, t₃=0.05 s;
   Quantity of pulses: 12 for 180 kW and respectively 35 and 48 pulses per 45 kW;
Productivity, linear speed of bottles: 0.4 m/s for 180 kW and 0.1 m/s for 45 kW. Length of the application zone of the field: 0.3 m; duration of application of the electric field pulses: 0.75 s;

Results:

| | | | Residual | Residual |
|---|---|---|---|---|
| | Speed of | Treatment | concentration | concentration |
| Electric | temperature | temperature | after tests | after tests |
| field | increase in | in | (units/ml) | (units/ml) |
| (V/cm) | ° C./s | ° C., +/−1° C. | Sacch. cer. | Asp. niger |
| 0 | 9 | 70 | 2.8 · 10¹ | 5 · 10² |
| 0 | 35 | 70 | <1 | <1 |
| 0 | 9 | 65 | 1.5 · 10³ | 1.8 · 10³ |
| 0 | 35 | 65 | <1 | <1 |
| 65 | 9 | 60 | 5.2 · 10¹ | 3.7 · 10¹ |
| 65 | 35 | 60 | <1 | <1 |
| 120 | 9 | 60 | 3-5 | 6-8 |
| 120 | 35 | 60 | <1 | <1 |
| 120 | 9 | 50 | 3.2 · 10⁴ | 2.2 · 10³ |
| 120 | 35 | 50 | 7.2 · 10¹ | 5-6 · 10¹ |
| 1020 | 9 | 50 | 2.7 · 10² | 1.0 · 10² |
| 1020 | 35 | 50 | <1 | <1 |
| 2540 | 9 | 45 | 3-5 | 1.1 · 10¹ |
| 2540 | 35 | 45 | <1 | <1 |

1-22. (canceled)

23. Process for the sterilisation or pasteurisation of a liquid to be treated, comprising heating the liquid to be treated by electric field waves having a frequency greater than 1 MHz, at a speed greater than 28° C. per second, to a treatment temperature T between 20° C. and 66° C., and according to the value of the treatment temperature T, exposure of the liquid to an alternating electric field in pulses immediately or slightly after the heating of the liquid, the amplitude E of the electric field in V/cm being selected such that the equation:
C(T)≦log(E+1)≦B(T)
is satisfied for the values:
B(T)=−2.340×10⁻⁵T³+1.290×10⁻³T²−3.110×10⁻²T+5.0
C(T)=−4.503×10⁻⁵T³+2.888×10⁻³T²−5.900×10⁻²T+4.0
where T is the treatment temperature in Celsius.

24. The process of claim 23, wherein the electric field alternates with an oscillation frequency between 100 kHz and 1000 kHz.

25. The process of claim 23 wherein the total calorific energy supplied to the liquid to be treated by said electric field pulse or pulses is less than 0.05 J/cm³.

26. The process of claim 23, wherein the application of one or more electric field pulses is carried out after the step of heating the liquid followed by a pause during which the electric field is zero or negligible and the temperature of the aqueous solution uniformises.

27. The process of claim 1, wherein the duration of application of an electric field pulse is between 10 and 100 microseconds and the repetition frequency of repition of the electric field pulses is between 10 and 100 Hz.

28. The process of claim 23, wherein the speed of heating is greater than 30° C. per second.

29. The process of claim 23, wherein the liquid to be treated is contained in a hermetically sealed container made of dielectric material.

30. The process of claim 29, wherein the container is transported by a transport liquid flowing in a conduit passing through heating and electric field application stations.

31. The process of claim 30, wherein the transport liquid has dielectric properties similar to those of the liquid to be treated.

32. The process of claim 29 or 30, wherein the transport liquid and the containers are agitated to make uniform the temperature of the transport liquid and the liquid to be treated.

33. A device for carrying out a process for the sterilisation of a liquid to be treated which is water-based or contains water, comprising a heating station with a system for heating liquids, a station for the generation of electric field with a system for the generation of electric field by pulses, and a transport device for transport of the liquid to be treated comprising a conduit capable of transporting a liquid, passing through the heating station and electrical field generating station, wherein the heating system comprises a wave generator operating at a frequency greater than 1 MHz and configured to heat all the liquid to be treated passing through the heating station at a rate greater than 28° C. per second, and in that the system for generation of electric fields by pulses is configured to generate an alternating electric field with an oscillation frequency between 100 kHz and 1000 kHz.

34. The device of claim 33 wherein the system for generation of electric field by pulses is configured to supply a total calorific energy of less than 0.05 J/cm³ to the liquid to be treated.

35. The device of claim 33, wherein the system for generation of electric field by pulses is configured to generate pulses having a duration between 10 and 100 microseconds.

36. The device of claim 33, comprising a cooling station downstream of the station for generation of electric field through which the transport device passes.

37. The device of claim 33, wherein the system for generation of electric field pulses comprises des electrodes arranged on either side of a cross-section of passage of the conduit and capable of generating an electric field transversal to this section.

38. The device of claim 33, wherein the system for generation of electric field pulses comprises an inductor with one or more primary windings arranged toroidally around a cross-section of passage of the conduit and capable of generating an electric field essentially longitudinal to this section.

39. The device of claim 33, comprising at least one electric field sensor in the zone of application of the electric field and temperature sensors along the transport device, upstream, downstream and in the heating station.

40. The device of claim 33, wherein the transport device comprises a pump system and a transport liquid for transporting containers containing the liquid to be treated along the conduit, and a return circuit for returning the transport liquid from an outlet to an inlet of the transport device.

41. The device of claim 33, wherein the transport liquid has dielectric properties similar to those of the liquid to be treated.

42. The device of claim 33, wherein the conduit comprises parts with different cross-sections of passage, intended to vary the flow speed of the transport liquid.

43. Use of a device according to any one of claims 39 to 42 for the decontamination of blood or a liquid blood component contained in hermetically sealed containers.

44. Use of a device according to any one of claims 39 to 42 for the sterilisation of drinks or liquid food products contained in hermetically sealed containers.
